(19) United States
(12) Patent Application Publication
(10) Pub. No.: US 2020/0362193 A1
(43) Pub. Date: Nov. 19, 2020

(54) ANTI-FOG COATINGS

(71) Applicant: Akita Innovations LLC, Billerica, MA (US)

(72) Inventors: Larry Takiff, Arlington, MA (US); Richard A. Minns, Arlington, MA (US); Matthew Easterbrooks, Rochester, MA (US)

(73) Assignee: Akita Innovations LLC, Billerica, MA (US)

(21) Appl. No.: 16/826,107

(22) Filed: Mar. 20, 2020

Related U.S. Application Data
(60) Provisional application No. 62/821,070, filed on Mar. 20, 2019.

(51) Int. Cl. C09D 151/10; C09D 133/08; C08F 283/06; C07C 271/24

(52) U.S. Cl. CPC C09D 151/10; C07C 271/24; C08F 283/06; C09D 133/08

(57) ABSTRACT

Anti-fog coatings and related articles, compositions, and methods are generally described. In some embodiments, an article may comprise a dual functional anti-fog and anti-fouling coating. The coating may be stimuli-responsive and the surface energy, and accordingly wettability, of the coating may reversibly change upon exposure to certain conditions. For instance, upon exposure to water, the coating may have a relatively high surface energy that allows water to wet the coating. Conversely, exposure to an oil may cause the coating to have a relatively low surface energy that repels the oil. In some embodiments, such a stimuli responsive coating may comprise a cross-linked polymer network with covalently attached oleophobic groups.

### RELATED APPLICATIONS

This application claims priority under 35 U.S.C. 119(e) to U.S. Provisional Application No. 62/821,070, filed Mar. 20, 2019, entitled “ANTI-FOG COATINGS”, which is incorporated herein by reference it its entirety.

### GOVERNMENT FUNDING

This invention was made with government support under W911QY-14-P-0244 and W911QY-16-C-0003 awarded by the Army SBIR and Army SBIR. The government has certain rights in the invention.

### TECHNICAL FIELD

Anti-fog coatings and related articles, compositions, and methods are generally described.

### BACKGROUND

In the presence of water vapor, condensation on a transparent or reflective surface results in a reduction or loss of transparency or reflectivity, respectively, due to either fogging from microscopic droplets of water or icing from freezing of condensed water. Anti-fogging agents and treatments have been developed to prevent the condensation of water on surfaces in the form of microscopic droplets by causing the condensed water to spread out into an even film that does not refract or scatter light. Anti-fogging agents and treatments work by coating the surface and causing the water to wet the surface by minimizing surface tension or maximizing surface energy. However, these anti-fogging coatings are susceptible to fouling that may disrupt or destroy the anti-fogging capabilities. Fouling is a problem in many applications, as oils and other organic matter have the propensity to preferentially coat and potentially ruin surfaces. Accordingly, improved articles, compositions, and methods are needed.

### SUMMARY

Anti-fog coatings are provided. The subject matter of the present invention involves, in some cases, interrelated products, alternative solutions to a particular problem, and/or a plurality of different uses of one or more systems and/or articles.

In one set of embodiments, articles are provided. In one embodiment, an article comprises a substantially transparent coating on a solid surface, wherein the coating comprises oleophobic groups covalently attached to a cross-linked polymer network, and wherein the water contact angle of the coating is less than or equal to about 20° and the hexadecane contact angle of the coating is greater than or equal to about 45°.

In another embodiment, an article comprises a substantially transparent coating on a solid surface, wherein the coating comprises oleophobic groups covalently attached to a cross-linked polymer network, wherein the article has a transmittance of at least about 80% for at least about 90 seconds under the standard EN 166:2001 clause 7.3.2.

In one embodiment, an article comprises a substantially transparent coating on a solid surface, wherein the coating comprises oleophobic groups covalently attached to a cross-linked polymer network via a hydrophilic linker, and wherein the coating has a first surface tension under a first set of conditions and a second surface tension under a second set of conditions.

In one set of embodiments, anti-fog coating compositions are provided. In one embodiment, an anti-fog coating composition comprises a multi-acrylate monomer, wherein the multi-acrylate monomer is hydrophilic; a monoacrylate monomer comprising an acrylate group, a hydrophilic linker, and a terminal oleophobic group; a hydrophilic monoacrylate monomer; and an initiator, wherein a weight percentage of the monoacrylate monomer comprising the acrylate group, the hydrophilic linker, and the terminal oleophobic group is between about 1 wt. % and about 10 wt. % of the anti-fog coating composition and wherein a weight percentage of solids in the anti-fog coating is greater than or equal to about 95 wt. %.

In one set of embodiments, compounds are provided. In one embodiment, a compound is of Formula I. In some embodiments, a compound of Formula I has the structure:

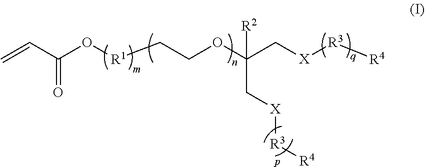

wherein:
each R¹ is independently —CR′₂—, —N(R″)—, —C(O)—, or —O—;
R² is hydrogen, halo, optionally substituted alkyl, optionally substituted fluoroalkyl, or —C(H)₂—X—(R³)_{q}—R⁴;
each R³ is independently —S(O₂)—, —CR′₂—, —N(R″)—, —S—, or —O—;
each R⁴ is independently C₁₋₆ fluoroalkyl;
each R′ is independently hydrogen, halo, optionally substituted alkyl, or optionally substituted haloalkyl;
each R″ is independently hydrogen, optionally substituted alkyl, or optionally substituted haloalkyl;
each X is independently —O—, —N(R″)—, or —S—;
each m, p, and q are independently 0, 1, 2, 3, 4, 5, 6, 7, or 8; and
n is an integer between 5 and 30.

In one embodiment, a compound is of Formula II. In some embodiments, a compound of Formula II has the structure:

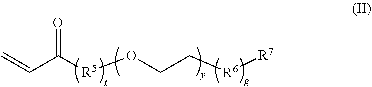

wherein:
each R⁵ is independently —CR*₂—, —N(R^{#})—, —C(O)—, optionally substituted carbocyclylene, or —O—;
each R⁶ is independently —CR*₂—, —N(R^{#})—, —S(O₂)—, —S—, or —O—;
each R⁷ is independently C₁₋₆ fluoroalkyl;
each R* is independently hydrogen, halo, optionally substituted alkyl, or optionally substituted haloalkyl;
each R^{#} is independently hydrogen, optionally substituted alkyl, or optionally substituted haloalkyl;
t and g are 0, 1, 2, 3, 4, 5, 6, 7, 8, 9, 10, 11, or 12; and
y is an integer between 5 and 30.

In one set of embodiments, methods are provided. In one embodiment, a method comprises applying a composition comprising cross-linking agents, agents comprising a terminal oleophobic group, agents comprising a hydrophilic moiety, and an initiator to a solid surface; activating the initiator; and allowing the cross-linking agents, the agents comprising a terminal oleophobic group, the agents comprising a hydrophilic moiety to polymerize on the solid surface to form a coating comprising a cross-linked polymer network.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D show schematics of a coating on a solid surface under different conditions, according to certain embodiments;

FIG. 2A shows a side view image of water and hexadecane droplets on a surface comprising a coated portion and an uncoated portion, according to one set of embodiments;

FIG. 2B shows a top view image of water and hexadecane droplets on a surface comprising a coated portion and an uncoated portion, according to certain embodiments;

FIG. 3A shows an image of a surface comprising a coated portion and an uncoated portion after exposure to steam, according to one set of embodiments; and

FIG. 3B shows an image of a surface comprising a coated portion and an uncoated portion after exposure to breath, according to one set of embodiments.

### DETAILED DESCRIPTION

Anti-fog coatings and related articles, compositions, and methods are generally described. In some embodiments, an article may comprise a dual functional anti-fog and anti-fouling coating. The coating may be stimuli-responsive and the surface energy, and accordingly wettability, of the coating may reversibly change upon exposure to certain conditions. For instance, upon exposure to water, the coating may have a relatively high surface energy that allows water to wet the coating. Conversely, exposure to an oil may cause the coating to have a relatively low surface energy that repels the oil. In some embodiments, such a stimuli-responsive coating may comprise a cross-linked polymer network with covalently attached oleophobic groups. The orientation of the oleophobic groups with respect to the surface of the cross-linked polymer network may reversibly change upon exposure to certain conditions. Under certain conditions, the orientation of the oleophobic groups may render the coating surface oleophobic. Under other conditions, the coating surface may be hydrophilic due, at least in part, to the orientation of the oleophobic groups and the surface characteristics of the cross-linked polymer network.

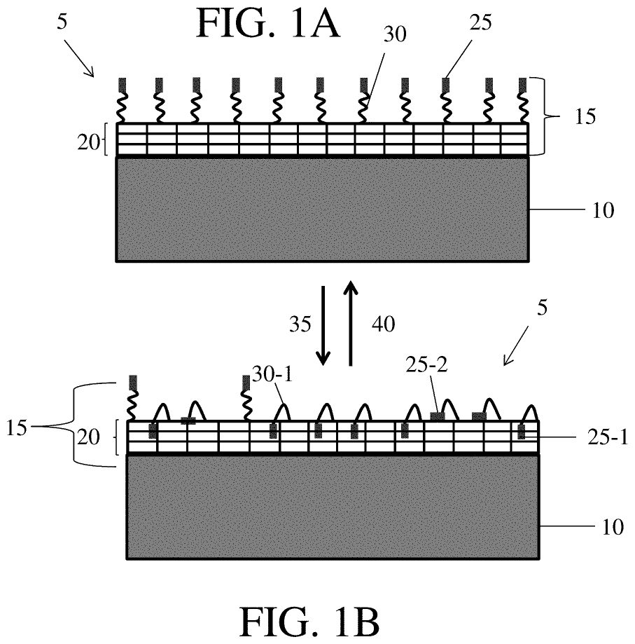

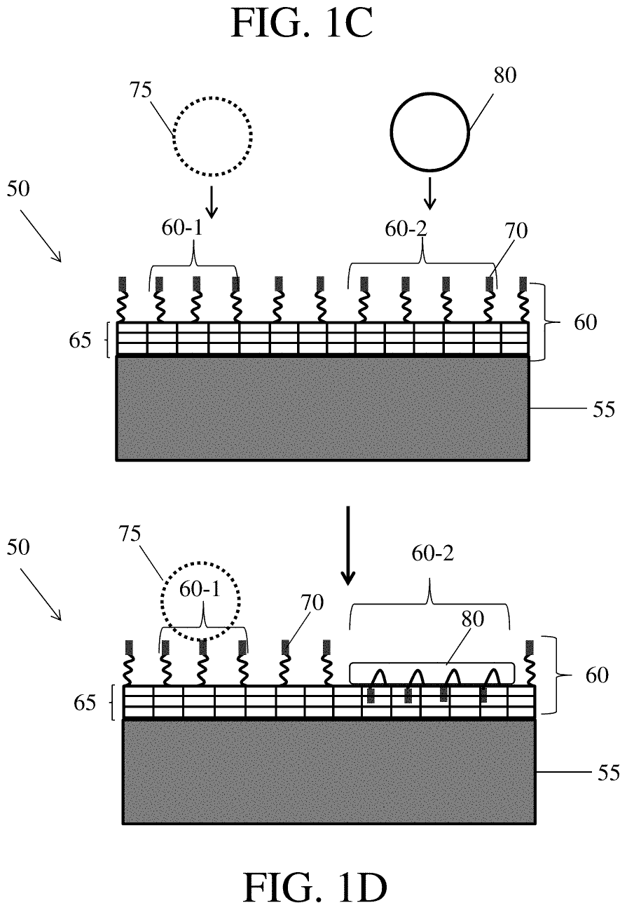

A non-limiting example of an article comprising a dual functional anti-fog and anti-fouling coating is shown in FIGS. 1A-1B. In some embodiments, an article 5 may comprise a solid surface 10 having a coating 15. In some instances, the surface and/or article may be substantially transparent or reflective. In certain instances, the surface and/or article may be substantially opaque. In some embodiments, the coating may comprise a cross-linked polymer network 20 and oleophobic groups 25. The oleophobic group 25 may be covalently attached to cross-linked polymer network 20. In some embodiments, as shown in FIGS. 1A-1B, the oleophobic groups may be indirectly covalently attached to the cross-linked network via a linker 30. The linker may be directly covalently bound to the cross-linked network. For example, the oleophobic groups may be covalently attached to the cross-linked network via a hydrophilic linker. In other embodiments, the oleophobic groups may be directly covalently attached to the cross-linked network. In some embodiments, the oleophobic groups may be a terminal group, as shown.

In some embodiments, the coating may be stimuli responsive, such that the surface energy of the coating reversibly changes under certain conditions. For instance, the coating may have a first surface energy under a first set of conditions (e.g., exposure to a lipophilic molecule, exposure to air), as illustrated in FIG. 1A, and exposure to a second set of conditions (e.g., exposure to a polar molecule, water), as indicated by arrow 35, may result in the coating having a second surface energy as illustrated in FIG. 1B. The first and second conditions may reversibly alter the surface energy, such that, e.g., exposure to the first condition again, as indicated by arrow 40, produces the first surface energy.

In some embodiments, the surface energy of the coating may depend, at least in part, on the orientation of the oleophobic groups with respect to the cross-linked network. Under certain conditions, as illustrated in FIG. 1A, at least a portion (e.g., substantially all) of the oleophobic groups may be oriented away from the cross-linked polymer network such that the oleophobic groups are exposed on the surface of the coating. In such embodiments, the surface concentration of oleophobic groups may be sufficient to render the surface oleophobic and/or cause the surface to have a relatively low surface energy. For example, in the presence of dry air or a lipophilic molecule (e.g., hexadecane, oil), the oleophobic groups may be oriented outward, such that the surface tension of the coating is less than or equal to about 25 mN/m and/or the minimum hexadecane contact angle is greater than or equal to about 45° (e.g., greater than or equal to about 50°, greater than or equal to about 75°, greater than or equal to about 90°).

Under different conditions, as illustrated in FIG. 1B, at least a portion (e.g., substantially all) of the oleophobic groups may oriented toward and/or within the cross-linked network such that the oleophobic groups are within the network or otherwise oriented to minimize exposure to the unfavorable component of the condition. For example, as illustrated in FIG. 1B, oleophobic group 25-1 is oriented within the cross-linked polymer network while oleophobic group 25-2 is positioned directly on the surface of the cross-linked polymer network. In some such embodiments, the surface characteristics of the polymer network may dictate, at least in part, the surface energy of the coating. For example, a polymer network having a relatively high hydrophilic content (e.g., a hydrophilic content greater than or equal to about 50 wt. %) may have a hydrophilic surface, a surface tension of greater than or equal to about 60 mN/m (e.g., greater than or equal to about 75 mN/m), and/or a maximum water contact angle of less than or equal to about 20°. In such cases, the orientation of the oleophobic groups toward and/or within the cross-linked polymer network may render the surface hydrophilic. In embodiments in which the oleophobic groups are covalently attached to the cross-linked polymer network via a hydrophilic linker, the hydrophilic linker may also contribute to the surface characteristics. For example, in the presence of water, the oleophobic groups may be oriented within and/or toward the cross-linked polymer network and the hydrophilic linkers (e.g., linker 30-1) may be more exposed to the outer surface of the coating as shown.

Without being bound by theory it is believed that the oleophobic groups rearrange upon exposure to certain conditions to obtain the most energetically favorable positions. The coating may be configured to provide sufficient flexibility to the oleophobic groups to allow for reorganization of the oleophobic groups, as described herein. For example, when the oleophobic groups are covalently attached to the cross-linked polymer network by a linker, the length of the linker may be selected to allow the oleophobic groups to stably orient toward and/or within the cross-linked polymer network and stably orient away from the cross-linked polymer network, such that the oleophobics are exposed on the surface. In certain embodiments, the cross-link density of the polymer network may be selected to allow for the oleophobic groups to stably orient within, toward, and/or away from the polymer network.

It is also believed that the composition of the cross-linked polymer network may influence, at least in part, the energetically favorable orientation of the oleophobic groups under certain conditions. Without being bound by theory, it is believed that surfaces minimize the free energy at their interfaces. For instance, when the hydrophilic content of the cross-linked polymer network is relatively high (e.g., greater than 50 wt. %) and the coating is substantially dry or exposed to certain lipophilic molecules, the energetically favorable orientation of at least a portion (e.g., substantially all) of the oleophobic groups may be away from the cross-linked polymer network. It is believed that this orientation minimizes the surface free energy. Conversely, the energetically favorable orientation of at least a portion (e.g., substantially all) of the oleophobic groups may be toward and/or within the cross-linked polymer network when the coating is exposed to polar molecules (e.g., hydrophilic molecules, water). It is believed that the oleophobic groups re-orient inward to expose hydrophilic groups in the cross-linked polymer network, thereby minimizing free energy at the water-polymer interface.

It should be understood that, in some embodiments, the orientation of the oleophobic groups may vary across the coating surface. In some embodiments, the orientation of an individual oleophobic group depends on the conditions that the individual oleophobic group experiences. That is, the surface energy of a first portion of the coating may be independent from a second portion of the coating, such that exposure of the first and second portions to different conditions may result in the first and second portions having different surface energies. For instance, a coating exposed to both water and oil may have portions of its surface that are oleophobic (e.g., having a surface tension of less than or equal to about 25 mN/m) and portions that are hydrophilic (e.g., surface tension of greater than or equal to about 60 mN/m, surface tension of greater than or equal to about 75 mN/m), depending on whether the portion is in contact with oil or water, respectively. In other embodiments, the surface energy of portions of the coating may not be independent of one another.

A non-limiting example of a coating having an oleophobic portion and a hydrophilic portion is shown in FIGS. 1C-D. As illustrated in FIG. 1C, an article 50 may comprise a solid surface 55 having a coating 60 including a cross-linked polymer network 65 and oleophobic groups 70. Prior to exposure to an oil droplet 75 and a water droplet 80, at least a portion (e.g., substantially all) of the oleophobic groups may be oriented away from the cross-linked polymer network, such that the overall coating surface is oleophobic (e.g., having a surface tension of less than or equal to about 25 mN/m). As illustrated in FIGS. 1C-1D, upon exposure to oil droplet 75, coating portion 60-1 may remain oleophobic, such that the oil droplet does not wet the surface. In some instances, the oil droplet may be shed from the coating surface. Upon exposure to water droplet 80, the oleophobic groups in coating portion 60-2 may reorient toward the cross-linked polymer network, such that the portion 60-2 is hydrophilic and the water wets portion 60-2. In such cases, different conditions on different portions of the coating may cause the orientation of oleophobic groups to vary across the surface.

In some embodiments, the coatings, described herein, may provide anti-fog and anti-fouling capabilities to an article while being mechanically robust. For instance, in the presence of fog and/or other forms of water vapor, the stimuli-responsive nature of the coating may prevent the condensation of water on surfaces in the form of microscopic droplets and allow the condensed water to spread out into an even film that does not refract or scatter light. In such cases, a transparent coated article may remain substantially transparent in the presence of fog and/or other forms of water vapor. The stimuli responsive nature may also allow the coating to be anti-fouling in the presence of lipophilic molecules (e.g., oils). For instance, in some embodiments, the response of the coating to lipophilic molecules may prevent the oil from wetting the surface, such that the lipophilic molecules have a relatively large contact angle (e.g., greater than or equal to about 45°), and are easily repelled from the surface upon application of a small amount of force. For example, the oil may roll-off of the coating due to the force of gravity or may be removed from the coating using relatively small shear forces. In some instances, the relatively large contact angle may allow the lipophilic molecules to be removed using an absorbent material. The coating may also be mechanically robust, such that the coating achieves at least the minimally accepted values for scratch and abrasion resistance as measured by test such as MIL-PRF-32432.

As described herein, a stimuli responsive anti-fog and anti-fouling coating may comprise a cross-linked polymer network with covalently attached oleophobic groups. The stimuli responsive nature of the coating may depend, at least in part, on certain features of the coating, such as the type of oleophobic groups, the weight percentage of the oleophobic groups, the cross-link density of the polymer network, the hydrophilic content of the cross-linked polymer network, linker length if applicable, linker type if applicable, and/or the location of the oleophobic groups, amongst other properties.

In some embodiments, the molecular weight between cross-links in the polymer network may be selected to allow for sufficient rearrangement of the oleophobic groups. For instance, in some embodiments, the molecular weight between cross-links may be at least about 100 g/mol, at least about 150 g/mol, at least about 200 g/mol, at least about 250 g/mol, at least about 300 g/mol, at least about 400 g/mol, at least about 500 g/mol, at least about 600 g/mol, at least about 700 g/mol, at least about 800 g/mol, or 900 g/mol and less than about 1,000 g/mol (e.g., less than about 700 g/mol, less than about 400 g/mol). In some embodiments, the molecular weight between cross-links may be between about 100 g/mol and about 1,000 g/mol (e.g., between about 200 g/mol and about 700 g/mol).

In some embodiments, the surface of the cross-linked polymer network absent the oleophobic groups and linkers, if applicable, may be hydrophilic. In some such cases, the cross-linked polymer network may have a relatively high hydrophilic content. In some embodiments, the hydrophilic content of the cross-linked polymer network is at least about 50 wt. %, at least about 55 wt. %, at least about 60 wt. %, at least about 65 wt. %, at least about 70 wt. %, at least about 75 wt. %, at least about 80 wt. %, at least about 85 wt. %, at least about 90 wt. % , or at least about 92 wt. % and less than or equal to about 99.5 wt. % (e.g., less than or equal to about 99 wt. %, less than or equal to about 98 wt. %, less than or equal to about 97 wt. %, less than or equal to about 96 wt. %, less than or equal to about 95 wt. %, less than or equal to about 94 wt. %, less than or equal to about 90 wt. %, less than or equal to about 85 wt. %, less than or equal to about 80 wt. %). In some embodiments, the hydrophilic content may be between about 50 wt. % and about 99.5 wt. % (e.g., between about 80 wt. % and about 99 wt. %, between about 90 wt. % and about 99 wt. %, between about 90 wt. % and about 95 wt. %). In some embodiments, the hydrophilic content may be between about 90 wt. % and about 99 wt. % (e.g., between about 90 wt. % and about 95 wt. %, about 93 wt. %). As used herein, hydrophilic content has its ordinary meaning in the art and refers to the weight percentage of hydrophilic monomers used to form the cross-linked polymer network.

In general, any suitable oleophobic group may be directly or indirectly covalently attached to the polymer network. In some embodiments, the oleophobic groups may be terminal groups that are capable of producing a coating having a surface tension of less than or equal to about 25 mN/m under certain conditions, such as exposure to lipophilic molecules (e.g., oil). In some embodiments, the oleophobic group may comprise one or more branched oleophobic moieties. In certain embodiments, oleophobic groups comprising branched oleophobic moieties provide anti-fog and/or anti-fouling performance that is superior to the performance exhibited by oleophobic groups that do not comprise a branched oleophobic moiety. In general, the oleophobic group may comprise any suitable number of branch points. For instance, in some embodiments, the oleophobic group may comprise one or more branch points, two or more branch points, three or more branch points, or four or more branch points. In some embodiments, at least some (e.g., one or more, two or more, three or more, four or more, all) of the branches in the oleophobic group are oleophobic. In some embodiments, the oleophobic group is non-linear. In certain instances, the oleophobic group is a linear group.

In some embodiments, the oleophobic group (e.g., branched oleophobic group) comprises one or more fluorine atoms. For instance, in some embodiments, the oleophobic group (e.g., branched oleophobic group) may comprise one or more fluorinated groups. For example, the oleophobic group (e.g., branched oleophobic group) may comprise one or more fluoroalkyl groups and/or heteroalkyl groups substituted with one or more fluoro or fluoroalkyl groups. In some instances, the oleophobic group may comprise one or more fluoroalkyl groups (perfluoroalkyl groups). In certain embodiments, the oleophobic group may comprise one or more heteroalkyl (e.g., alkoxy) groups substituted with one or more fluoro or fluoroalkyl groups. In some such embodiments, the heteroalkyl group may be perfluorinated. In certain embodiments, the oleophobic group may comprise one or more perfluorinated groups.

In some embodiments, the oleophobic group includes at least one fluorine atom. In some instances, the oleophobic group may be highly fluorinated. For instance, in some embodiments, the oleophobic group may comprise a fluorine to hydrogen ratio of, for example, at least about 0.2:1, at least about 0.5:1, at least about 1:1, at least about 2:1, at least about 5:1, or at least about 10:1. In some cases, the oleophobic group (e.g., branched oleophobic group) comprises a perfluorinated group, i.e., the group contains fluorine atoms but contains no hydrogen atoms. In some such embodiments, the oleophobic group comprises one or more groups (e.g., branches) of formula —CₙF₂ₙ₊₁, where n is an integer greater than or equal to 1 (e.g., greater than 1). In some cases, n is an integer less than or equal to 10 and greater than or equal to about 2. In some instances, n may be between 2 and 8 (e.g., between 3 and 6). In certain embodiments, the oleophobic group may comprise two or more, three or more, or four or more groups of formula —CF₂ₙ₊₁, as described herein. In some embodiments, the oleophobic group may comprise one or more groups (e.g., branches) of formula —O—CₙF₂ₙ₊₁, where n is an integer greater than or equal to 1 (e.g., greater than 1). In some cases, n is an integer less than or equal to 10 and greater than or equal to about 2. In some instances, n may be between 2 and 8 (e.g., between 3 and 6). In certain embodiments, the oleophobic group may comprise two or more, three or more, or four or more groups of formula —O—CₙF₂ₙ₊₁, as described herein. In some embodiments, the oleophobic group (e.g., branched oleophobic group) may comprise one or more heteroatoms (e.g., oxygen) and one or more fluorinated groups.

In some embodiments, the weight percentage of oleophobic groups in the coating is between about 0.5 wt. % and about 10 wt. %, between about 0.5 wt. % and about 8 wt. %, between about 0.5 wt. % and about 6 wt. %, between about 0.5 wt. % and about 5 wt. %, between about 1 wt. % and about 10 wt. %, between about 1 wt. % and about 8 wt. %, between about 2 wt. % and about 10 wt. %, between about 2 wt. % and about 8 wt. %, between about 2 wt. % and about 6 wt. %, or between about 4 wt. % and about 6 wt. %.

As noted above, in some embodiments, the oleophobic groups are covalently attached to the cross-linked polymer network via a hydrophilic linker. In certain embodiments, the hydrophilic linker is covalently bonded to the cross-linked network. In general, the hydrophilic linker may be any suitable molecule (e.g., oligomer, polymer) that allows for rearrangement of the oleophobic groups into stable orientations.

In some embodiments, the hydrophilic linker may be a polymer. As used herein, the term “polymer” has its ordinary meaning in the art and may refer to a macromolecule formed from one or more monomers that comprises at least three repeat units. In some embodiments, the hydrophilic linker comprises between about 5 repeat units and about 25 repeat units (e.g., between about 5 repeat units and about 22 repeat units, between about 5 repeat units and about 20 repeat units, between about 5 repeat units and about 18 repeat units, between about 10 repeat units and about 25 repeat units, between about 10 repeat units and about 22 repeat units, between about 10 repeat units and about 20 repeat units, between about 15 repeat units and about 25 repeat units, between about 15 repeat units and about 20 repeat units). In certain embodiments, the hydrophilic linker comprises between about 10 repeat units and about 25 repeat units (e.g., between about 10 repeat units and about 20 repeat units, between about 15 repeat units and about 25, between about 15 repeat units and about 20 repeat units). In some embodiments, the hydrophilic linker comprises ethylene glycol repeat units.

In some embodiments, the length of the hydrophilic linker is at least about 1 times and less than about 10 times, at least about 2 times and less than about 8 times, or at least about 3 times and less than about 5 times the length of the oleophobic group.

In some embodiments, the coating may comprise particles (e.g., nanoparticles). The particles may serve to increase the scratch resistance and/or hardness of the coating without adversely affecting the transparency of the coating. In general, any suitable particle that imparts beneficial scratch resistance and/or hardness properties of the coating without adversely affecting the transparency or coating formation process may be used. In some embodiments, the particles may be silica particles. In some cases, the silica particles may have an average diameter of less than about 1,000 nm (e.g., nanoparticles. For instance, in some embodiments, the average diameter of the particles (e.g., silica particles) may be less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 100 nm, or less than or equal to about 100 nm.

In some embodiments, the particles (e.g., silica particles) may be attached to the cross-linked polymer network. In certain embodiments, the particles may be covalently attached to the cross-linked polymer network. In some such embodiments, at least a portion of the surface of the particles may be modified to comprise one or more functional groups (e.g., polymerizable functional groups). For instance, prior to inclusion in the cross-linked polymer network, a particle may be modified to have acrylate groups on at least a portion of its surface. In some such embodiments, at least a portion of the functional groups (e.g., acrylate groups) on the particles may react with one or more components of the coating to form a covalent bond between the particles and components (e.g., monomers) in the coating. In other embodiments, the particles are attached to the coating by a non-covalent bond.

In some embodiments, the weight percentage of particles (e.g., silica particles) in the coating is between about 0.5 wt. % and about 10 wt. %, between about 0.5 wt. % and about 8 wt. %, between about 0.5 wt. % and about 6 wt. %, between about 0.5 wt. % and about 5 wt. %, between about 0.5 wt. % and about 4 wt. %, between about 0.5 wt. % and about 3 wt. %, between about 0.5 wt. % and about 2 wt. %, or between about 0.5 wt. % and about 1.5 wt. %. In some embodiments, the weight percentage of particles (e.g., silica particles) in the coating is between about 0.5 wt. % and about 2 wt. % (e.g., between about 0.5 wt. % and about 1.5 wt. %, about 1 wt. %).

As described herein, the coating may have anti-fogging and anti-fouling properties. In some such embodiments, the coating may have a water contact angle of less than or equal to about 20° (e.g., less than or equal to 18°, less than or equal to 15°, less than or equal to 12°, less than or equal to 10°, less than or equal to 8°, less than or equal to 5°) and/or a hexadecane contact angle of greater than or equal to about 45° (e.g., greater than or equal to about 60°, greater than or equal to about 75°, greater than or equal to about 90°, greater than or equal to about 105°, greater than or equal to about 120°). Exposure to air or lipophilic molecules (e.g., hexadecane) may cause the coating to have a surface tension less than or equal to about 25 mN/m (e.g., less than or equal to about 20 mN/m) and exposure to hydrophilic molecules (e.g., water) may cause the coating to have a surface tension greater than or equal to 75 mN/m (e.g., greater than or equal to about 80 mN/m). In some embodiments, a hexadecane contact angle of 45° may correlate to a surface tension of about 20-25 mN/m. In some embodiments, upon exposure to air or lipophilic molecules, the coating may have a surface tension similar to known hydrophobic material, such as polytetrafluoroethylene which has a surface tension of about 20 mN/m. In some embodiments, the difference in the surface tension of the coating when exposed to a first set of conditions (e.g., lipophilic molecules) and a second set of conditions (e.g., hydrophilic molecules) may be between about 50 mN/m and about 100 mN/m.

In some embodiments, a coated transparent article may remain substantially transparent when exposed to water vapor (e.g., fog). For instance, in some embodiments, the coated transparent article may have a transmittance of at least about 80% for at least about 30 seconds (e.g., at least about 45 seconds, at least about 60 seconds, at least about 75 seconds, at least about 90 seconds, at least about 105 seconds, at least about 120 seconds, at least about 135 seconds, at least about 150 seconds, at least about 165 seconds, at least about 180 seconds, at least about 4 minute, at least about 5 minute, at least about 8 minute, at least about 10 minute) under the standard EN 166:2001 clause 7.3.2 test.

In some embodiments, the coating may have a pencil hardness of 3B. As used herein, pencil hardness refers to a standard test of coating hardness as measured by an Elcometer model 501 Pencil Hardness tester (Elcometer Inc., Rochester Hills, Mich.).

In some embodiments, the coating may have a substantially uniform thickness (e.g., wherein the thickness of the material does not vary more than 25%, more than 20%, more than 15%, more than 10%, more than 5%, or more than 1% over the surface of the article). In some cases, the thickness may not be substantially uniform. In some embodiments, the coating may have a thickness of between about 100 nm and about 100 microns, between about 250 nm and about 100 microns, between about 500 nm and about 100 microns, between about 1 micron and about 100 microns, or between about 1 micron and about 50 microns. The thickness of the coating may be determined using profilometry or interferometry.

In general, the coating may be formed on any suitable surface (e.g., solid surface) of an article. In some embodiments, the article and/or a surface of the article may have an optical transmission of greater than or equal to about 80% (e.g., greater than or equal to about 85%, greater than or equal to about 90%, greater than or equal to about 95%, 100%). In such cases, the coating may have an optical transmission of greater than or equal to about 80% (e.g., greater than or equal to about 85%, greater than or equal to about 90%, greater than or equal to about 95%, 100%). Non-limiting examples of suitable articles and/or surfaces include glass, polycarbonate, polyamide, polyimides, polyurethanes, polystyrene, polysulfones, polyolefins, polyketones, polyesters (e.g., poly(ethylene terephthalate)), polyethers, poly(alkylene oxides), polyacrylates (e.g., poly(methyl methacrylate)), modified cellulose (e.g., cellulose acetate propionate (CAP), cellulose acetate butyrate (CAB)), metals (e.g., silver, aluminum), metalloids (e.g., silicon, germanium), zinc sulfide, zinc selenide, spinel, diamond, sapphire, and combinations thereof. In some embodiments, the article and/or a surface of the article may be a polymer (e.g., thermoplastic or thermoset). Suitable polymers are described in the Polymer Handbook, Fourth Ed. Brandrup, J. Immergut, E. H., Grulke, E. A., Eds., Wiley-Interscience: 2003, which is incorporated herein by reference in its entirety. In certain embodiments, the article and/or a surface of the article may be a metal or metalloid.

In some embodiments, the coating is adhered to the surface via non-covalent or covalent bonds. In some cases, the coating is adhered to the surface via non-covalent bonds such as hydrogen bonds, ionic bonds, dative bonds, and/or Van der Waals interactions. The coating and the surface may comprise functional groups capable of forming such bonds. It should be understood that covalent and non-covalent bonds between components may be formed by any type of reactions, as known to those of ordinary skill in the art, using the appropriate functional groups to undergo such reactions. Chemical interactions suitable for use with various embodiments described herein can be selected readily by those of ordinary skill in the art, based upon the description herein.

In another aspect, methods are provided. In some embodiments, the stimuli-responsive coating, described herein, may be formed by applying a polymerizable coating composition to a surface (e.g., solid surface) of an article and polymerizing the coating composition on the surface of the article. The coating composition may comprise cross-linking agents (e.g., multi-acrylate monomers), agents comprising a terminal oleophobic group (e.g., monoacrylate monomers comprising a terminal oleophobic group), agents comprising a hydrophilic moiety (e.g., hydrophilic monomer), an initiator, and optionally particles (e.g., silica particles). In some embodiments, the coating composition may comprise a relatively low weight percentage of or no fluid carrier. In certain embodiments, at least a portion of the surface of the particles comprise functional groups (e.g., polymerizable groups, acrylate groups, monomers). As used herein, the term “polymerization” has its ordinary meaning in the art and may refer to the process of converting monomers into polymers and/or a three-dimensional network (e.g., cross-linked polymer network).

The initiator may be activated on the surface of the article via any suitable means, including exposure to heat, moisture, or electromagnetic radiation, to induce polymerization and/or cross-linking. After activation of the initiator, the coating composition may be exposed to conditions that facilitate polymerization (e.g., temperature). The degree of polymerization and/or cross-linking may be dictated, at least in part, by controlling the reaction time, reaction temperature, concentration of initiator, concentration of cross-linking agent, and concentration of other agents in the coating compositions, amongst other. In some embodiments, the in situ polymerization of the coating composition on a surface of an article may result in a coating comprising a cross-linked polymer network, as described herein, without further modification (e.g., chemical reactions).

Activation of the initiator and/or polymerization may be carried out for any suitable period of time. In some embodiments, a suitable length of time is determined by whether a substantial portion of the starting material has been transformed into the desired product, for example, by using simple screening tests known to those of ordinary skill in the art. For example, completeness of polymerization may be determined by measuring coating hardness, scratch resistance, solvent resistance, or loss of acrylate C═C in the IR spectrum. In some cases, activating and/or polymerizing are carried out for less than about 60 seconds, about 45 seconds, about 30 seconds, about 15 seconds, about 5 seconds, about 1 second, about 45 milliseconds, about 30 milliseconds, or about 15 milliseconds. In some cases, the period of time is between about 1 millisecond and about 1 second, between about 1 millisecond and about 45 milliseconds, between about 1 millisecond and about 30 milliseconds, or between about 5 millisecond and about 30 milliseconds.

In embodiments in which electromagnetic radiation is used to activate an initiator and/or facilitate polymerization, the wavelength and/or intensity at which the polymerization is conducted and/or the initiator is activated may be selected as desired. For example, a coating may be polymerized by a single exposure or multiple exposures to a xenon flashlamp. As another example, a coating may be polymerized by exposure to continuous-wave (cw) light from a mercury lamp or other CW source. Using simple screening tests, those of ordinary skill in the art will be able to select appropriate wavelengths and/or intensities for polymerization and/or activation. In some embodiments, the electromagnetic radiation may be in the ultraviolet region.

In general, any suitable coating method may be used to apply the coating composition to a surface of the article. The coating may be applied using techniques known to those of ordinary skill in the art. For example, a coating may be applied by a spin-casting method, a drop-casting method, a dip coating method, a roll coating method, a flow coating method, a screen coating method, a spray coating method, a screen printing method, an ink-jet method, and the like. In some embodiments, the coating composition may be applied to a surface via spray coating.

In some embodiments, a conformal coating may be formed on a surface using the methods, described herein. As used herein, a “conformal” coating refers to a coating formed on and attached or adhered to a material, wherein the coating physically matches the exterior contour of the surface area of the underlying material and the coating does not substantially change the macroscopic morphology of the underlying material. In some instances, the coating does not substantially change the microscopic morphology of the underlying material. That is, the coated material has a morphology (e.g., macroscopic, microscopic) that is essentially the same as the morphology of an essentially identical material lacking the coating, under essentially identical conditions. It should be understood that the conformal coating may uniformly increase one or more dimensions (e.g., thickness) of the material, however, the overall morphology of the material remains essentially unchanged. Additionally, the conformal coating may form a stable structure and may not delaminate from the surface of the article. In some cases, the conformal coating may be substantially free of defects and/or voids, and may uniformly coat the underlying material, or portion thereof.

As noted above, in some embodiments, the coating composition may be formulated such that a stimuli responsive coating, as described here, may be formed via in situ polymerization without further modification (e.g., chemical reactions). In certain embodiments, the components of the coating composition, weight percentage of the components, and/or ratio of certain components to one another may be selected to produce the desired coating. For instance, in some embodiments, the coating composition may comprise multi-acrylate monomers, monoacrylate monomers comprising a terminal oleophobic group, and an initiator in a fluid carrier or without a fluid carrier.

In some embodiments, the coating composition may comprise a relatively low weight percentage of fluid carrier. For instance, in some embodiments, the weight percentage of the fluid carrier in the coating composition may be less than or equal to about 50 wt. %, less than or equal to about 40 wt. %, less than or equal to about 30 wt. %, less than or equal to about 20 wt. %, less than or equal to about 10 wt. %, less than or equal to about 5 wt. %, less than or equal to about 4 wt. %, less than or equal to about 2 wt. %, less than or equal to about 1 wt. %, less than or equal to about 0.5 wt. %, less than or equal to about 0.2 wt. %, less than or equal to about 0.1 wt. %, less than or equal to about 0.05 wt. %, less than or equal to about 0.01 wt. %, or less than or equal to about 0.005 wt. %. In some embodiments, the weight percentage of the fluid carrier in the coating composition may be less than or equal to about 5 wt. % (e.g., less than or equal to about 1 wt. %, less than or equal to about 0.5 wt. %, less than or equal to about 0.1 wt. %). In certain embodiments, the weight percentage of the fluid carrier in the coating composition may be less than or equal to about 0.1 wt. % (e.g., less than or equal to about 0.05 wt. %, less than or equal to about 0.01 wt. %). In some embodiments, the coating composition may comprise 0 wt. % of a fluid carrier.

In some embodiments, when present, the fluid carrier is a liquid (e.g., organic solvent). In some instances, the liquid may have a relatively high boiling point. For example, the liquid may have a boiling point of greater than or equal to about 60° C. (e.g., greater than or equal to about 70° C., greater than or equal to about 80° C., greater than or equal to about 100° C., greater than or equal to about 110° C., greater than or equal to about 120° C., greater than or equal to about 130° C.). In general, a liquid fluid carrier does not distort (e.g., soften, swell) and/or affect the transparency of the surface to be coated. In certain embodiments, liquid fluid carriers include alcohols (e.g., ethanol, isopropyl alcohol, diacetone alcohol), ethyl acetate, 2-butanone, dimethyl carbonate, 1-methoxy-2propanol, propylene glycol methyl ether acetate, ethyl lactate, or combinations thereof.

As used herein, the term “solids” with respect to a coating composition refers to the components in the coating composition that are not a fluid carrier. In some instances, some solids may be dissolved in a fluid carrier and in other instances, at least a portion (e.g., substantially all) of the solids may not be dissolved in the fluid carrier. In certain embodiments, the primary function of the solids is not to dissolve or otherwise carry the components in the composition. In some embodiments, the weight percentage of solids in the coating composition may be greater than or equal to about 50 wt. %, greater than or equal to about 60 wt. %, greater than or equal to about 70 wt. %, greater than or equal to about 80 wt. %, greater than or equal to about 90 wt. %, greater than or equal to about 92 wt. %, greater than or equal to about 95 wt. %, greater than or equal to about 97 wt. %, greater than or equal to about 98 wt. %, or greater than or equal to about 99 wt. %. In some embodiments, the solids in the coating composition may comprise multi-acrylate monomers, monoacrylate monomers comprising a terminal oleophobic group, and an initiator. In some embodiments, the coating may comprise 100 wt. % solids. In such cases, the coating may not contain a fluid carrier.

As used herein, the term “multi-acrylate monomer” refers to a monomer having two or more acrylate groups. Non-limiting examples of multi-acrylate monomers include diacrylate monomers, triacrylate monomers, tetraacrylate monomers, pentaacrylate monomers, hexaacrylate monomers, and combinations thereof. In some embodiments, the multi-acrylate monomers in the coating composition may be the same type (e.g., diacrylate monomers, triacrylate monomers) and/or have the same chemical structure. In some instances, the multi-acrylate monomers in the coating composition may be the same type, but have a different chemical structure. For instance, the multi-acrylate monomers may consist of at least two triacrylate monomers having different chemical structures.

In certain embodiments, the multi-acrylate monomers may be different in type. For example, the multi-acrylate monomers may include diacrylate monomers and triacrylate monomers. In one embodiment, the multi-acrylate monomers may include diacrylate monomers, triacrylate monomers, and tetraacrylate monomers. As another example, the multi-acrylate monomers may include triacrylate monomers and tetraacrylate monomers.

In some instances, when the multi-acrylate monomers in the coating composition are of a different type (e.g., diacrylate monomers, triacrylate monomers, tetraacrylate monomers), a monomer type may consist of monomers having the same chemical structure and/or a monomer type may consist of monomers having different chemical structures. For example, a coating composition may include diacrylate monomers, triacrylate monomers, and/or tetraacrylate monomers and each monomer type (e.g., diacrylate, triacrylate) has the same chemical structure. In another example, a coating composition including diacrylate monomers, triacrylate monomers, and/or tetraacrylate monomers may include two or more different diacrylate monomers, two or more different triacrylate monomers, and/or two or more different tetraacrylate monomers.

In general, any suitable multi-acrylate monomer may be used. Non-limiting examples of multi-acrylate monomer include poly(ethylene glycol) diacrylate, ethylene glycol diacrylate, trimethylolpropane triacrylate, di(trimethylolpropane) tetraacrylate, ethoxylated trimethylolpropane triacrylate, pentaerythritol tetraacrylate, trimethylolpropane propoxylate triacrylate, epoxy acrylates, polyester acrylates, and urethane acrylates. In some embodiments, one or more multi-acrylate monomers may comprise a hydrophilic polymer. In some embodiments, one or more multi-acrylate monomers may be hydrophilic. Those of ordinary skill in the art would be aware of suitable multi-acrylate monomers based on the description herein.

In some embodiments, a multi-acrylate monomer may comprise two or more ethylene glycol repeat units. In some embodiments, a multi-acrylate monomer may comprise between about 5 and about 25 (e.g., between about 5 and about 22, between about 5 and about 20, between about 5 and about 18, between about 10 and about 25, between about 10 and about 22, between about 10 and about 20, between about 15 and about 25, between about 15 and about 20) ethylene glycol repeat units. In certain embodiments, the multi-acrylate monomer comprises between about 10 and about 25 (e.g., between about 10 and about 20, between about 15 and about 25, between about 15 and about 20) ethylene glycol repeat units.

In some embodiments, the weight percentage of multi-acrylate monomers may be greater than or equal to about 25 wt. %, greater than or equal to about 30 wt. %, greater than or equal to about 40 wt. %, greater than or equal to about 50 wt. %, greater than or equal to about 60 wt. %, greater than or equal to about 70 wt. % and, e.g., less than or equal to about 100 wt. % (e.g., less than or equal to about 98 wt. %, less than or equal to about 95 wt. %, less than or equal to about 92 wt. %, less than or equal to about 90, less than or equal to about 85 wt. %, less than or equal to about 80 wt. %) of the coating composition. In some embodiments, the weight percentage of multi-acrylate monomers may be between about 25 wt. % and about 100 wt. %, between about 30 wt. % and about 100 wt. %, between about 40 wt. % and about 100 wt. %, between about 50 wt. % and about 100 wt. %, between about 50 wt. % and about 90 wt. %, between about 60 wt. % and about 90 wt. %, or between about 70 wt. % and about 85 wt. % of the composition.

In some embodiments, regardless of the weight percentage and/or mole percentage of acrylate monomers comprising an oleophobic groups in the compositions, the ratio of acrylate monomers comprising an oleophobic group to all other acrylate monomers in the composition is between about 1:50 and about 1:5, between about 1:30 and about 1:5, between about 1:30 and about 1:10, and between about 1:20 and about 1:15.

In general, any suitable monoacrylate or multiacrylate monomer comprising a terminal oleophobic group may be used. In some embodiments, the acrylate monomer may comprise a terminal oleophobic group (e.g., perfluorohexyl) and a hydrophilic linker (e.g., polyethylene glycol). In some embodiments, one or more acrylate may include a terminal oleophobic group as described herein. In some embodiments, a monoacrylate monomer comprising an oleophobic group is a compound of Formula I:

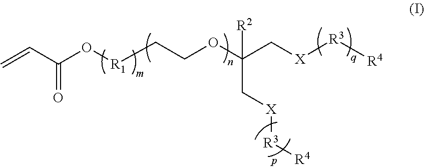

wherein:
each R¹ is independently —CR′₂—, —N(R″)—, —C(O)—, or —O—;
R² is hydrogen, halo, optionally substituted alkyl, optionally substituted fluoroalkyl, or —C(H)₂—X—(R³)_{q}—R⁴;
each R³ is independently —S(O₂)—, —CR′₂—, —N(R″)—, —S—, or —O—;
each R⁴ is independently C₁₋₆ fluoroalkyl;
each R′ is independently hydrogen, halo, optionally substituted alkyl, or optionally substituted haloalkyl;
each R″ is independently hydrogen, optionally substituted alkyl, or optionally substituted haloalkyl;
each X is independently —O—, —N(R″)—, or —S—;

each m, p, and q are independently 0, 1, 2, 3, 4, 5, 6, 7, or 8; and
n is an integer between 5 and 30.

In some embodiments, m is less than or equal to 5. In some cases, m is 0. In certain instances m is greater than zero. For instance, m may be 1, 2, 3, 4, 5, 6, 7, or 8 or m may be 1, 2, 3, 4, or 5. In certain embodiments in which m is greater than zero, —(R¹)ₘ— may comprise —C(O)—O—, —CR′₂—CR′₂—, and/or —N(R″)—C(O)—. For instance, in some embodiments in which m is 5, —(R¹)ₘ— is —CR′₂—CR′₂—N(R″)—C(O)—O—. In some such cases, —(R¹)ₘ— is —CH₂—CH₂—N(H)—C(O)—O—.

In some embodiments, n is an integer between 5 and 25, between 5 and 20, between 10 and 30, between 10 and 25, between 10 and 20, or between 15 and 20.

In some embodiments, R² is hydrogen, halo, optionally substituted alkyl, or optionally substituted fluoroalkyl. For instance, R² is hydrogen, halo, or optionally substituted alkyl. In some cases, R² is hydrogen. In certain embodiments, R² is fluoroalkyl or —C(H)₂—X—(R³)_{q}—R⁴. In some such cases, R² is —C(H)₂—X—(R³)_{q}—R⁴. In such cases, X, R³, q, and R⁴ may be as described herein.

In certain embodiments, each X is independently —O— or —N(R″)—. In some such cases, X is —O—. In other cases, X is —N(R″)—. In some instances, each X is independently —O— or —S—. For example, X is —S—. In certain instances, each X is independently —S— or —N(R″)—. In certain cases, in which one or more X is —N(R″)—, each R″ is independently hydrogen or optionally substituted alkyl. In some cases, R″ is hydrogen. In some instances, R″ is optionally substituted alkyl (e.g., methyl, ethyl, optionally substituted C₁₋₆ alkyl, optionally substituted C₁₋₃ alkyl). For example, R″ may be CH₃.

In some embodiments, p and/or at least one q (e.g., each q) are less than or equal to about 6 (e.g., less than or equal to about 5, less than or equal to about 3). In some instances, p and/or at least one q (e.g., each q) are independently 0, 1, 2, 3, 4, or 5. In certain embodiments, p and/or at least one q (e.g., each q) is zero. In some embodiments, p and/or at least one q (e.g., each q) are greater than zero. For instance, p and at least one q (e.g., each q) may be greater than zero. In some such cases, p and at least one q (e.g., each q) are independently 1, 2, 3, 4, 5, 6, 7, or 8. In certain instances, p and at least one q (e.g., each q) are independently 1, 2, 3, 4, or 5. In certain instances, p and at least one q (e.g., each q) are independently 1, 2, or 3.

In some embodiments, each R³ is independently —S(O₂)—, —CR′₂—, or —O—. For instance, in some embodiments when p is greater than zero, —(R³)ₚ— may comprise —CR′₂—CR′₂—O—. In some such cases, R′ is hydrogen, halo (e.g., fluoro), or optionally substituted haloalkyl (e.g., optionally substituted C₁₋₃ haloalkyl, optionally substituted C₁₋₃ fluoroalkyl, —CF₃). For instance, —(R³)ₚ— may comprise —CH₂—C(F)(CF₃)—O—. In some instances in which p is greater than zero, —(R³)ₚ— comprises —S(O₂)—. In some embodiments in which at least one q is greater than zero, —(R³)_{q}— may be as described herein for —(R³)ₚ—. In some instance, in which p and at least one q is greater than zero, each —(R³)_{q}— has the same chemical structure as —(R³)ₚ—. In other cases, at least one —(R³)_{q}— has a different chemical structure than —(R³)ₚ—. In certain cases, each —(R³)_{q}— has the same chemical structure. In other cases, each —(R³)_{q}— does not have the same chemical structure.

In certain embodiments, each R⁴ is independently C₁₋₆ perfluoroalkyl (e.g., C₂₋₆ perfluoroalkyl, C₃₋₆ perfluoroalkyl, C₂₋₅ perfluoroalkyl). In some instances, each R⁴ is independently C₂₋₆ fluoroalkyl (e.g., C₃₋₆ fluoroalkyl, C₂₋₅ fluoroalkyl).

In some instances, each R′ is independently hydrogen, halo (e.g., fluoro), or optionally substituted haloalkyl (optionally substituted C₁₋₃ haloalkyl, optionally substituted C₁₋₃ fluoroalkyl, optionally substituted C₁₋₃ perfluoroalkyl, —CF₃). In some cases, each R″ is independently hydrogen or optionally substituted alkyl.

In some embodiments, the compound of Formula I is selected from the group consisting of:

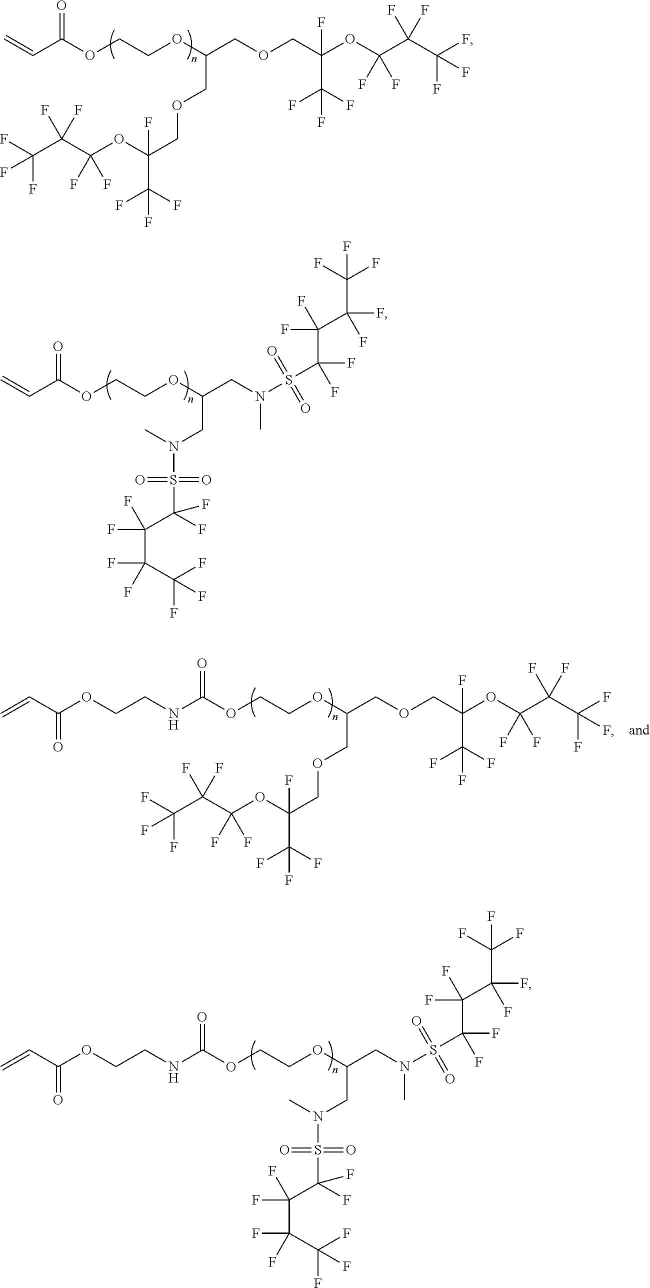

wherein n is as described herein.

In some embodiments, a monoacrylate monomer comprising an oleophobic group is a compound of Formula II:

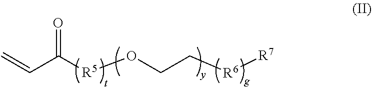

wherein:
each R⁵ is independently —CR*₂—, —N(R^{#})—, —C(O)—, optionally substituted carbocyclylene, or —O—;
each R⁶ is independently —CR*₂—, —N(R^{#})—, —S(O₂)—, —S—, or —O—;
each R⁷ is independently C₁₋₆ fluoroalkyl;
each R* is independently hydrogen, halo, optionally substituted alkyl, or optionally substituted haloalkyl;
each R^{#} is independently hydrogen, optionally substituted alkyl, or optionally substituted haloalkyl;
t and g are 0, 1, 2, 3, 4, 5, 6, 7, 8, 9, 10, 11, or 12; and
y is an integer between 5 and 30.

In some embodiments, t is less than or equal to 10 (e.g., less than or equal to 8, less than or equal to 5). In some cases, t is 0. In certain instances t is greater than zero. For instance, t may be 1, 2, 3, 4, 5, 6, 7, or 8 or t may be 1, 2, 3, 4, or 5. In certain embodiments in which t is greater than zero, —(R⁵)ₜ— may comprise —O—, —CR*₂—CR*₂, optionally substituted carbocyclylene, —C(O)—N(R^{#})—, and/or —N(R^{#})—C(O)—. For instance, in some embodiments in which t greater than zero, —(R⁵)ₜ— is —O—CR*₂—CR*₂—N(R^{#})—C(O)—. In some such cases, —(R⁵)ₜ— is —O—CH₂—CH₂—N(H)—C(O)—. As another example, in some embodiments in which t greater than zero, —(R⁵)ₜ— is —O—CR*₂—CR*₂—O—C(O)—N(R^{#})— optionally substituted carbocylene —CR*₂—N(R^{#})—C(O)—.

In some embodiments, y is an integer between 5 and 25, between 5 and 20, between 10 and 30, between 10 and 25, between 10 and 20, or between 15 and 20.

In some embodiments, g is less than or equal to about 10 (e.g., less than or equal to about 8, less than or equal to about 5, less than or equal to about 3). In some instances, g is 0, 1, 2, 3, 4, or 5. In certain cases, g is 0, 1, 2, or 3. In certain embodiments, g is zero. In some embodiments, g is greater than zero. In some such cases, g is 1, 2, 3, 4, 5, 6, 7, or 8. In certain instances, g is 1, 2, 3, 4, or 5. In certain instances, g is 1, 2, or 3.

In some embodiments, each R⁶ is independently —CR*₂—, —N(R^{#})—, —S(O₂)—, or —O—. For instance, in some embodiments when g is greater than zero, —(R⁶)_{g}— may comprise —N(R^{#})—S(O₂)—. In some such cases, R^{#} is hydrogen or optionally substituted alkyl (e.g., methyl, optionally substituted C₁₋₃ alkyl). For instance, —(R⁶)_{g}— may comprise N(CH₃)—S(O₂)—. In some instances in which g is greater than zero, —(R⁶)_{g} comprises —O—CR*₂—. In some such cases, R* is hydrogen or halo (e.g., fluoro). For instance, —(R⁶)_{g}— may comprise —O—CH₂—.

In certain embodiments, R⁷ is independently C₁₋₆perfluoroalkyl (e.g., C₂₋₆ perfluoroalkyl, C₃₋₆ perfluoroalkyl, C₂₋₅ perfluoroalkyl). In some instances, R⁷ is independently C₂₋₆ fluoroalkyl (e.g., C₃₋₆ fluoroalkyl, C₂₋₅ fluoroalkyl). In some instances, each R* is independently hydrogen or halo (e.g., fluoro). In some cases, each R^{#} is independently hydrogen or optionally substituted alkyl.

In some embodiments, the compound of Formula II has the structure:

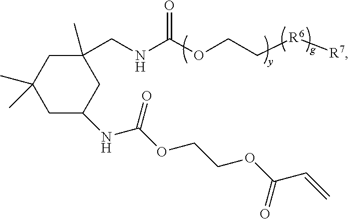

wherein y, R⁶, g, and R⁷ are as described herein.

In some embodiments, the compound of Formula II is selected from the group consisting of:

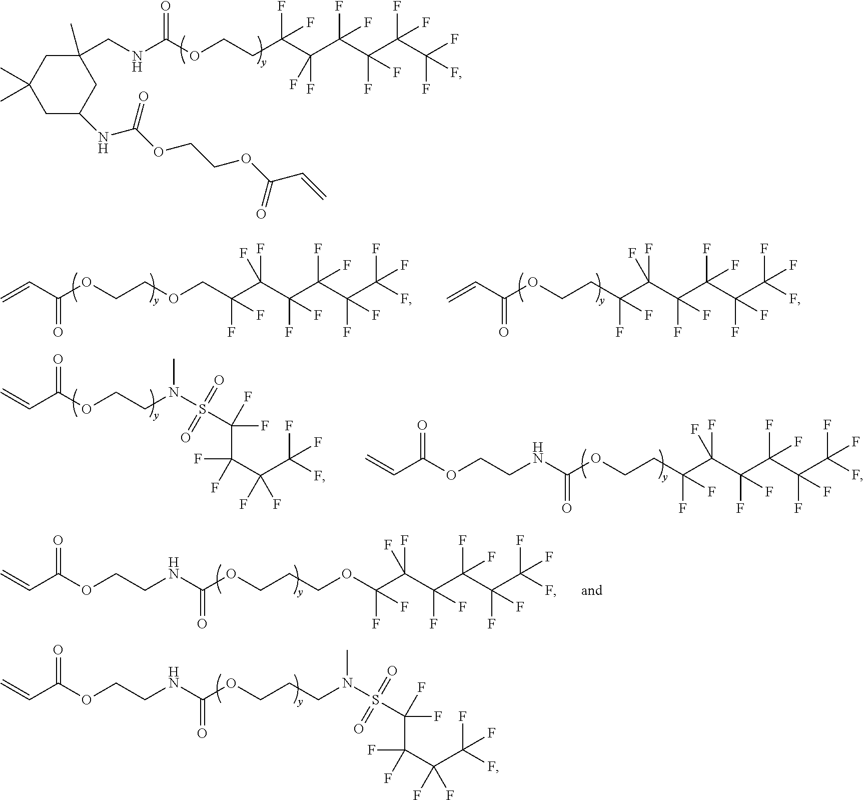

wherein y is as described herein.

In some embodiments, the weight percentage of monomers comprising a terminal oleophobic group may be between about 1 wt. % and about 30 wt. %, between about 1 wt. % and about 15 wt. %, between about 1 wt. % and about 10 wt. %, between about 1 wt. % and about 6 wt. %, between about 2 wt. % and about 30 wt. %, between about 2 wt. % and about 15 wt. %, between about 2 wt. % and about 10 wt. %, between about 2 wt. % and about 6 wt. %, or between about 3 wt. % and about 6 wt. % of the coating composition. In some embodiments, the weight percentage of monomers comprising a terminal oleophobic group is between about 2 wt. % and about 6 wt. % (e.g., between about 3 wt. % and about 6 wt. %, about 5 wt. %) of the coating composition.

In some embodiments, the coating composition may comprise a monoacrylate monomer that does not comprise an oleophobic group. In some such embodiments, the monoacrylate monomer may be hydrophilic. For example, the hydrophilic monoacrylate may comprise two or more ethylene glycol repeat units. In some embodiments, the hydrophilic monoacrylate may comprise between about 5 and about 25 (e.g., between about 5 and about 22, between about 5 and about 20, between about 5 and about 18, between about 10 and about 25, between about 10 and about 22, between about 10 and about 20, between about 15 and about 25, between about 15 and about 20) ethylene glycol repeat units. In certain embodiments, the hydrophilic monoacrylate monomer comprises between about 10 and about 25 (e.g., between about 10 and about 20, between about 15 and about 25, between about 15 and about 20) ethylene glycol repeat units.

In some embodiments, the weight percentage of the hydrophilic monoacrylate may be between about 1 wt. % and about 30 wt. %, between about 2 wt. % and about 30 wt. %, between about 5 wt. % and about 30 wt. %, between about 10 wt. % and about 30 wt. %, between about 1 wt. % and about 25 wt. %, between about 1 wt. % and about 20 wt. %, between about 2 wt. % and about 25 wt. %, between about 2 wt. % and about 20 wt. %, between about 5 wt. % and about 20 wt. %, or between about 10 wt. % and about 20 wt. % of the coating composition. In some embodiments, the weight percentage of the hydrophilic monoacrylate is between about 10 wt. % and about 20 wt. % of the coating composition.

In general, any suitable initiator may be used. In some embodiments, the initiator is a free radical initiator. For instance, the initiator may be a photoinitiator that undergoes, e.g., photolysis to produce a free radical. In some instances, the initiator is a thermal initiator that undergoes, e.g., thermal decomposition to produce a free radical. In some embodiments, the weight percentage of the initiator may be between about 0.1 wt. % and about 3 wt. %, between about 0.1 wt. % and about 2.5 wt. %, between about 0.1 wt. % and about 2 wt. %, between about 0.1 wt. % and about 1.5 wt. %, between about 0.5 wt. % and about 3 wt. %, between about 0.5 wt. % and about 2.5 wt. %, or between about 0.5 wt. % and about 2 wt. % of the coating composition. In some embodiments, the weight percentage of the initiator may be between about 0.5 wt. % and about 2 wt. % (e.g., between about 0.5 wt. % and about 1.5 wt. %, about 1 wt. %) of the coating composition.

In some embodiments, the coating composition may optionally comprise particles having a surface comprising functional groups (e.g., polymerizable groups, acrylate groups, monomers). In some embodiments, the weight percentage of such particles (e.g., silica particles) in the coating composition is between about 0.5 wt. % and about 10 wt. %, between about 0.5 wt. % and about 8 wt. %, between about 0.5 wt. % and about 6 wt. %, between about 0.5 wt. % and about 5 wt. %, between about 0.5 wt. % and about 4 wt. %, between about 0.5 wt. % and about 3 wt. %, between about 0.5 wt. % and about 2 wt. %, or between about 0.5 wt. % and about 1.5 wt. %. In some embodiments, the weight percentage of particles (e.g., silica particles) in the coating composition is between about 0.5 wt. % and about 2 wt. % (e.g., between about 0.5 wt. % and about 1.5 wt. %, about 1 wt. %).

As used herein, the terms “wet” and “wetting” has its ordinary meaning in the art and may refer to the process in which a drop of liquid spreads over a solid or liquid surface. In this process, the interface between the surface and a gas is replaced by an interface between the same surface and the liquid from the drop.

As used herein, the term “substantially transparent” has its ordinary meaning in the art and may refer to a material (e.g., article, coating, surface) that has an optical transmission of greater than or equal to about 80%. In some instances, a substantially transparent material may have an optical transmission between about 80% and about 100%, between about 85% and about 100%, between about 90% and about 100%, or between about 95% and about 100%.

As used herein, a “set of conditions” or “conditions” may comprise, for example, a particular solvent, chemical reagent, type of atmosphere (e.g., nitrogen, argon, oxygen, air etc.), temperature, pH, electromagnetic radiation, or combinations thereof. Some embodiments may involve a set of conditions comprising exposure to solvent or type of atmosphere.

As used herein, the terms “attached”, “bound”, “bonded”, and “adhered” refer to attachment or adhesion via covalent bonds or non-covalent bonds (e.g., ionic bonds, van der Waals forces, etc.).

As used herein, the term “oleophobic” (also known as “lipophobic”) has its ordinary meaning in the art. When the term “oleophobic” is used to refer to a surface, the term has its ordinary meaning in the art and may refer to a surface having a hexadecane contact angle of greater than or equal to about 45°. In some embodiments, an oleophobic surface may have a lower surface energy than a given oil. For instance, an oleophobic surface may have a surface energy of less than or equal to about 25 mNm. In some embodiments, oleophobic molecules and/or groups have a tendency to not readily dissolve in fat-like solvents, such as lipids and certain non-polar solvents (e.g., hydrocarbons). In certain embodiments, oleophobic molecules and/or groups may also be hydrophobic.

As used herein, the term “lipophilic” has its ordinary meaning in the art and when referring to molecules and/or groups may refer to molecules and/or groups that have a tendency to readily dissolve in fat-like solvents, such as lipids and certain non-polar solvents (e.g., hydrocarbons). In some embodiments, the lipophilic molecule is non-polar.

As used herein, the term “hydrophilic” has it ordinary meaning in the art and when referring to a surface may refer to a surface that has a water contact angle of less than 90°. When the term “hydrophilic” is used with respect to a molecules and/or groups, the term has its ordinary meaning in the art and may refer to molecules and/or groups that have a tendency to interact with polar solvents, in particular with water, or with other polar groups. One of ordinary skill in the art would be able to readily select hydrophilic molecules and/or groups based on general knowledge in the art and the disclosure herein.

“Weight percentage”, as used herein, refers to the dry weight percentage.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the anti-fog and anti-fouling applications. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. Examples of haloalkyl groups include —CF₃, —CF₂CF₃, —CF₂CF₂CF₃, —CCl₃, —CFCl₂, —CF₂Cl, and the like.

As used herein, “fluoroalkyl” is a substituted alkyl group as defined herein wherein one or more of the hydrogen atoms are independently replaced by a fluorine. “Perfluoroalkyl” is a subset of fluoroalkyl, and refers to an alkyl group wherein all of the hydrogen atoms are independently replaced by a fluorine. In some embodiments, the fluoroalkyl moiety has 1 to 8 carbon atoms (“C₁₋₈ fluoroalkyl”). In some embodiments, the fluoroalkyl moiety has 1 to 6 carbon atoms (“C₁₋₆ fluoroalkyl”). In some embodiments, the fluoroalkyl moiety has 1 to 4 carbon atoms (“C₁₋₄ fluoroalkyl”). In some embodiments, the fluoroalkyl moiety has 1 to 3 carbon atoms (“C₁₋₃ fluoroalkyl”).

In some embodiments, the fluoroalkyl moiety has 1 to 2 carbon atoms (“C₁₋₂ fluoroalkyl”). Examples of fluoroalkyl groups include —CF₃, —CF₂CF₃, —CF₂CF₂CF₃, and the like.

As used herein, “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “halo” or “halogen” refers to fluorine (fluoro, —F), chlorine (chloro, —Cl), bromine (bromo, —Br), or iodine (iodo, —I).

Affixing the suffix “-ene” to a group indicates the group is a divalent moiety, e.g., alkylene is the divalent moiety of alkyl, heteroalkylene is the divalent moiety of heteroalkyl, and acylene is the divalent moiety of acyl. In some instances, a substituted amino may be divalent.

As understood from the above, alkyl, carbocyclyl, haloalkyl, and fluoroalkyl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” carbocyclyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})2, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)3—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl), —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The following examples are intended to illustrate certain embodiments of the present invention, but do not exemplify the full scope of the invention.

### EXAMPLE 1

This example describes the synthesis of HEA-IPDI-HEA and HEA-IPDI-FPEG that could be used to prepare a UV-cured transparent coating with a low water contact angle (e.g., anti-fogging) and a high hexadecane contact angle (e.g., anti-fouling).

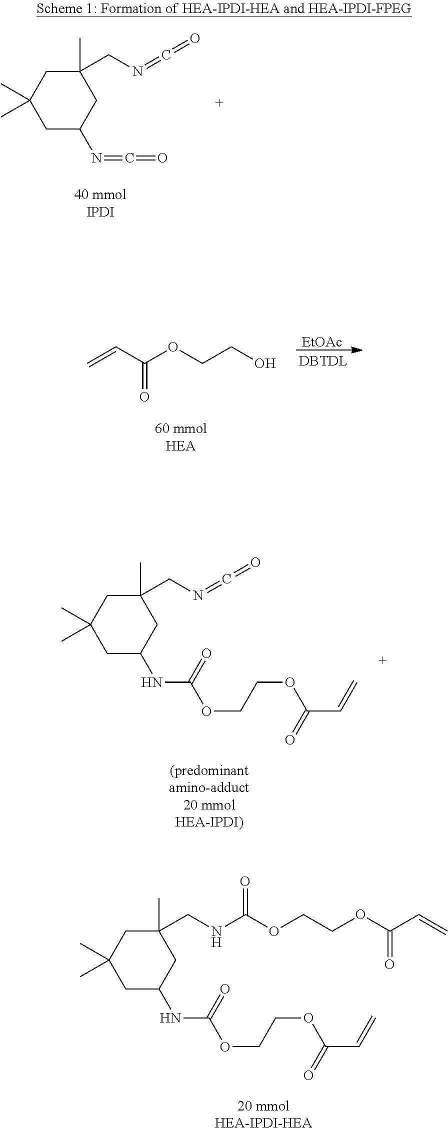

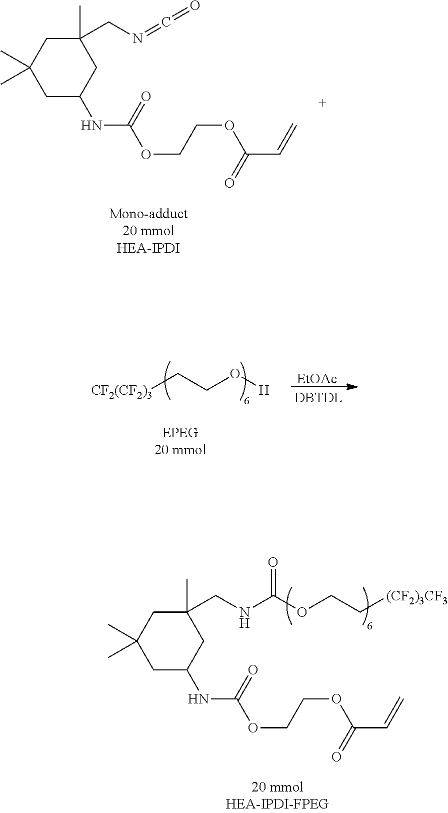

HEA-IPDI-HEA and HEA-IPDI-FPEG were formed as shown in Scheme 1. Briefly, to a 50-ml 3NRB flask equipped with a magnetic stir egg, a thermometer, an addition funnel, and a drying tube filled with 4 Å molecular sieves was added isophorone diisocyanate IPDI (8.4 ml), dibutyltin dilaurate (DBTDL, 90 mg), and ethyl acetate (EtOAc, 9 ml). A solution of 2-hydroxyethyl acrylate (HEA, 6.3 ml), mono methyl ether of hydroquinone (MEHQ, 32 mg), and EtOAc (9 ml) was placed in the addition funnel and added dropwise with stirring at 300 rpm. The flask was set in an oil bath at room temperature, and the temperature of the reaction solution increased from 24° C. to <35° C. due to the reaction exotherm. After addition was complete in about 40 minutes, the oil bath was heated to 40° C. and the solution was stirred for about 21 hours at 300 rpm and 40° C. under dry air. The mixture of HEA-IPDI-HEA and HEA-IPDI was not separated prior to use.

DuPont Capstone® FS-3100 (10.55 g) and EtOAc (9 ml) were added to the mixture of HEA-IPDI-HEA and HEA-IPDI in the 50-ml flask. Stirring of the solution was continued at 300 rpm and 40° C. for about 19 hours.

All of the resulting HEA-IPDI-HEA and HEA-IPDI-FPEG mixture was used to prepare a coating composition by adding the photoinitiator, Darocur 4265 (265 mg), and more EtOAc to a total formulation weight of 50.0 g comprising about 52% solids. A clear solution was obtained upon mixing that comprised the components shown in Table 1 plus the EtOAc solvent. Weight percentages shown in Table 1 are based on the total solids without EtOAc.

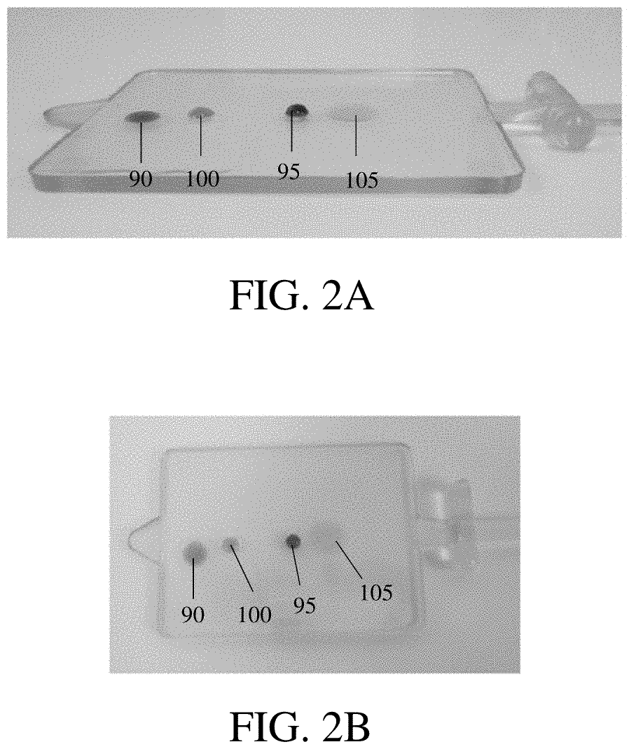

This composition was puddle-cast on a polycarbonate plaque obtained from Revision Military Technologies and spread to a rectangle covering about ⅓ of the area. After brief drying, the coating was UV-cured with 5 flashes from a xenon flashlamp. Droplets (10 μl each) of water (blue) and hexadecane (orange) were applied to both the coated and uncoated areas as shown in FIG. 2A (side view) and FIG. 2B (top-down view). The water droplet 90 spread out on the coated portion, but water droplet 95 beaded up on the uncoated portion. As expected, the hexadecane droplet 105 spread out on the uncoated portion, but hexadecane droplet 100 beaded up on the coated portion, thereby showing that the coated surface exhibited both hydrophilic and oleophobic properties. The coated surface was not fogged with a breath test or with water vapor over a water bath heated to 50° C., whereas the uncoated surface immediately fogged. Also, a hexadecane drop on the coated surface was readily washed away with distilled water to re-form a clean surface, whereas the hexadecane drop on the uncoated surface left a smudge when washed with water.

TABLE 1
Solids in the Coating Composition

| | Weight % | |
|---|---|---|
| Component Structure: | (Approximate) | Component Names: |
| 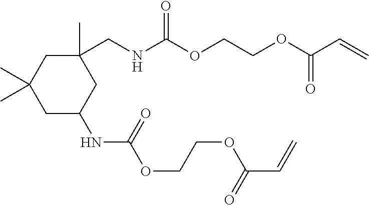 [SMILES: C=CC(=O)OCCOC(=O)NCC1(C)CC(NC(=O)OCCOC(=O)C=C)CC(C)(C)C1] | 31% | HEA-IPDI-HEA |
| 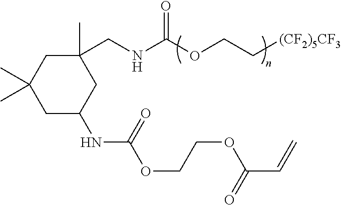 | 68% | HEA-IPDI-FPEG |
| 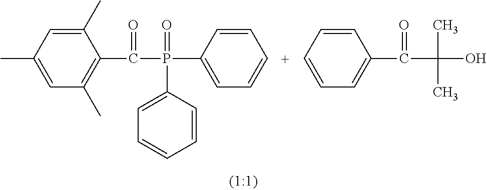 | 1% | Darocur 4265, BASF 50111614 1:1 Blend of Darocur 1173 and TPO |

### EXAMPLE 2

This example describes a transparent, dual functional anti-fog and anti-fouling coating on a transparent substrate.

The coating composition was prepared by adding the solid components shown in Table 2 together. The coating composition contained about 15% ethyl acetate, and a mixture of HEA-IPDI-HEA and HEA-IPDI-FPEG was prepared as described in Example 1. The composition was then vortexed, degassed, and sonicated. The weight percentages shown in Table 2 were based on the total dry solids.

TABLE 2
Solids in the Coating Composition

| | Weight % | |
|---|---|---|
| Component Structure: | (Approximate) | Component Names: |
| 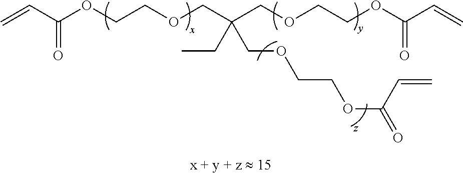 | 72% | Ethoxylated(15) trimethylolpropane triacrylate Sartomer SR9035 (EO₁₅TMPTA) |
| 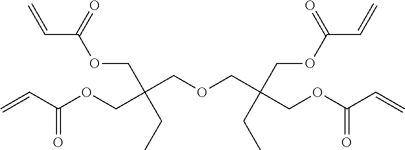 [SMILES: C=CC(=O)OCC(CC)(COCC(CC)(COC(=O)C=C)COC(=O)C=C)COC(=O)C=C] | 8% | Di-trimethylolpropane tetraacrylate Sartomer SR355 (DTMPTTA) |
| 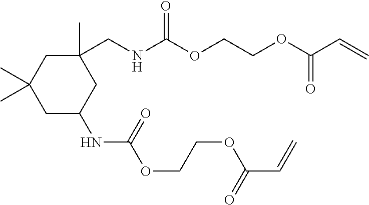 [SMILES: C=CC(=O)OCCOC(=O)NCC1(C)CC(NC(=O)OCCOC(=O)C=C)CC(C)(C)C1] | 6% | HEA-IPDI-HEA |
| 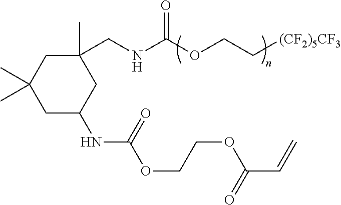 | 13% | HEA-IPDI-FPEG |
| 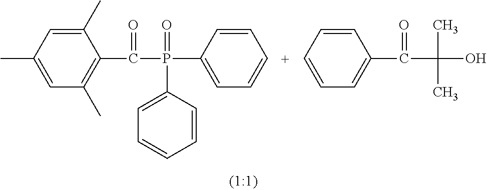 | 1% | Darocur 4265, BASF 50111614 1:1 Blend of Darocur 1173 and TPO |

After sonication, several drops of the coating solution were puddle-cast on a polycarbonate substrate (Lexan, General Purpose, 3×3×⅛-inch), spread to an approximate 35 mm square, allowed to dry briefly, and UV-cured with 5 flashes from a xenon flashlamp.

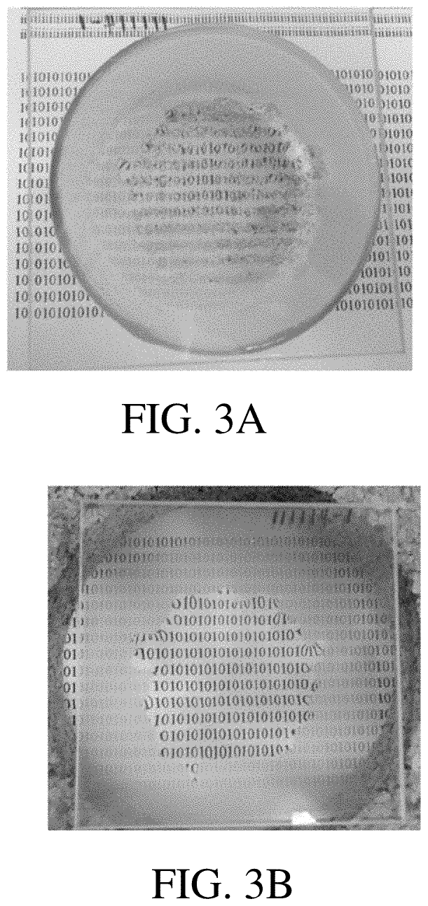

As shown in FIG. 3A, a steam test using a water bath at 40° C. fogged only the uncoated portion, leaving the coated portion clear. The steam test involved setting the polycarbonate, coated side down, on the rim of a Pyrex dish (70×50 mm) that was about a third filled with water heated to 50° C. on a hot plate. FIG. 3A shows a photograph of the polycarbonate substrate above a piece of white paper containing black numbers after the steam test.

As shown in FIG. 3B, the coated portion passed a breath test without fogging while the adjacent uncoated portion fogged. The breath test consisted of exhaling air on the substrate for about 2 seconds from a distance of about 2 cm. FIG. 3B shows a photograph of the polycarbonate substrate on a cork ring 3 cm above a piece of white paper containing black numbers after the breath test. The picture was taken immediately after the substrate was fogged with breath.

The coated polycarbonate substrate was substantially transparent and exhibited antifog properties, oleophobicity, good hardness, and good adhesion to polycarbonate.

### Example 3

This example describes the formation of a transparent anti-fogging and anti-fouling coating from a solvent free coating composition. The coating also comprised silica nanoparticles.

### Synthesis of Active Components

Pure Merck Advanced Fluorosurfactant 182 (5.0 g) was dissolved in dichloromethane (5.6 g) with dibutyltin dilaurate (0.0030 g) and 4-methoxyphenol (0.0012 g). 2-Isocyanatoethyl acrylate (0.589 g, IEA) was added dropwise over 30 minutes with stirring at ambient temperature. After the complete IEA addition, the reaction was stirred for 30 minutes at ambient temperature, then heated to 35° C. for 60 minutes, and then allowed to stir at ambient temperature overnight. The reaction was quenched with anhydrous 2-propanol (0.250 mL).

Pure polyethylene glycol monomethyl ether-750 (5.0 g) was dissolved in dichloromethane (6.2 g) with dibutyltin dilaurate (0.0056 g) and 4-methoxyphenol (0.0023 g). 2-Isocyanatoethyl acrylate (1.129 g) was added dropwise over 30 minutes with stirring at ambient temperature. After the complete IEA addition, the reaction was stirred for 30 minutes at ambient temperature, heated to 35° C. for 60 minutes, and then allowed to stir at ambient temperature overnight. The reaction was quenched with anhydrous 2-propanol (0.480 mL).

Acrylated silica nanoparticles were generated by reacting an acrylated silyl coupling agent with colloidal silica nanoparticles. To generate the acrylate silane coupling agent, O-(acryloxyethyl)-N-(triethoxysilylpropyl) carbamate (HEA-IPTES), 3-isocyanatopropyltriethoxysilane (4.95 g) was added dropwise to a neat solution of 2-hydroxyethyl acrylate (2.33 g) and dibutyltin dilaurate (0.025 g) over a period of 10 minutes with magnetic stirring. The reaction was stirred at room temperature for 2 hours. A portion of the HEA-IPTES (1.09 g) was added dropwise to a solution of colloidal silica nanoparticles in 2-propanol (10.0 g, 30% SiO₂ by wt.) and distilled water (0.2 mL) over 5 minutes with continuous stirring at room temperature. Once the addition was complete, the reaction was heated to 60° C. and stirred over the weekend.

### Formulation Generation

A formulation consisting of 78.21% ethoxylated (15) trimethylolpropane triacrylate, 14.85% polyethylene glycol monomethyl ether urethane acrylate, 4.95% Merck Advanced Fluorosurfactant 182 urethane acrylate, 1.00% acrylated silica nanoparticles, and 0.99% Darocur 4265 by wt. was prepared by combining the ingredients using a mini-vortexer. Residual solvent was removed using a rotary evaporator.

TABLE 3
Solid Components in the Coating Composition

| | Weight % | |
|---|---|---|
| Component Structure | (Approximate) | Component Name |
| 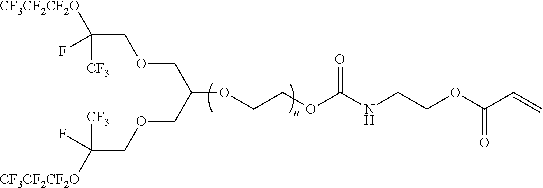 | 5% | Merck Advanced Fluorosurfactant 182 Urethane Acrylate (avg n is 17) |
| 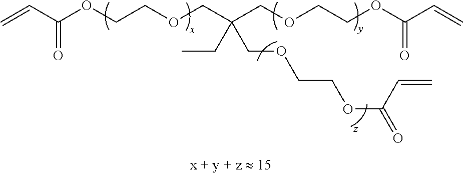 | 78% | Ethoxylated (15) trimethylolpropane triacrylate (Sartomer SR9035) |
| 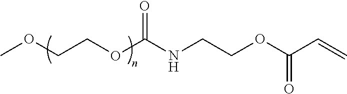 | 15% | Methoxy Polyethylene Glycol 750 Urethane Acrylate (n is 16-17) |
| 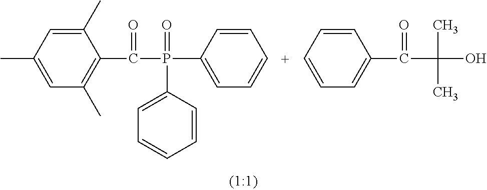 | 1% | Darocur 4265 |
| — | 1% | Acrylated-Silica nanoparticles |

### Coating Procedure

A solvent-free formulation was spray coated onto cleaned substrates with a commercial air brush using a dry gas propellent, held in an oxygen free environment for a minimum of 10 minutes, and then cured with 5 flashes from 640W-s Xenon flash lamp yielding a smooth, non-tacky surface not easily scratched with a fingernail and exhibiting 3B pencil hardness.

Cured coatings prepared from this formulation exhibited excellent antifog performance with optical transmittance maintained above 80% for more than 120 seconds under the standard EN 166:2001 clause 7.3.2 test. These coatings also exhibited complete fogging resistance for 180 seconds over 50° C. and 60° C. steam baths in testing similar to that shown in FIG. 3A (testing was terminated at 180 seconds).

Oil and water droplet tests were performed by placing 10 μl droplets of hexadecane, olive oil, and distilled water on the coatings of this example. The hexadecane and olive oil droplets beaded up and did not change dimensions over an observation time of 5 minutes. However, water droplets fully wetted the coatings within 60 seconds. Hexadecane and olive oil droplets were completely removed from the coatings simply by rinsing with a stream of water. A commercial antifog coating that does not contain any oleophobic groups was fully wetted with water droplets, but hexadecane and olive oil also spread out on that coating and were not readily rinsed off with water.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a substantially transparent coating on a solid surface, wherein the coating comprises oleophobic groups covalently attached to a cross-linked polymer network, wherein the article has a transmittance of at least about 80% for at least about 90 seconds under the standard EN 166:2001 clause 7.3.2, and wherein the water contact angle of the coating is less than or equal to about 20° and the hexadecane contact angle of the coating is greater than or equal to about 45°.

2. (canceled)

3. The article of claim 1, wherein the oleophobic groups are covalently attached to the cross-linked polymer network via a hydrophilic linker.

4. (canceled)

5. The article of preceding claim 1, wherein the coating further comprises silica particles.

6. The article of claim 1, wherein the silica particles are covalently attached to the cross-linked polymer network.

7-10. (canceled)

11. The article of claim 1, wherein hydrophilic linker comprises ethylene glycol repeat units.

12. The article of claim 1, wherein the hydrophilic linker comprises between about 5 and about 20 repeat units.

13. The article of claim 1, wherein the oleophobic groups are branched.

14. The article of claim 1, wherein the oleophobic groups comprise fluorinated groups.

15. The article of claim 1, wherein the oleophobic groups are terminal groups.

16. (canceled)

17. The article of claim 1, wherein the hydrophilic content of the cross-linked polymer network is at least about 50 wt. %.

18-21. (canceled)

22. The article of claim 1, wherein the article has a transmittance of at least about 80% for at least about 90 seconds under the standard EN 166:2001 clause 7.3.2.

23-31. (canceled)

32. An anti-fog coating composition, comprising:
a multi-acrylate monomer, wherein the multi-acrylate monomer is hydrophilic;
a monoacrylate monomer comprising an acrylate group, a hydrophilic linker, and a terminal oleophobic group;
a hydrophilic monoacrylate monomer; and
an initiator,
wherein a weight percentage of the monoacrylate monomer comprising the acrylate group, the hydrophilic linker, and the terminal oleophobic group is between about 1 wt. % and about 10 wt. % of the anti-fog coating composition and wherein a weight percentage of solids in the anti-fog coating is greater than or equal to about 95 wt. %.

33. The composition of claim 32, further comprising silica nanoparticles, wherein acrylate groups are on at least a portion of a surface of the silica nanoparticles.

34-35. (canceled)

36. The composition of claim 32, wherein a weight percentage of the hydrophilic monoacrylate monomers is between about 10 wt. % and about 20 wt. %.

37. The composition of claim 32, wherein the initiator is a free radical photoinitiator.

38. A compound of Formula I having the structure: 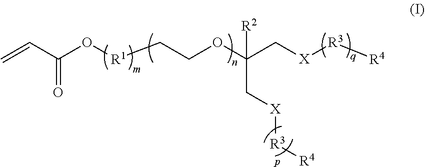
wherein:
each R¹ is independently —CR′₂—, —N(R″)—, —C(O)—, or —O—;
R² is hydrogen, halo, optionally substituted alkyl, optionally substituted fluoroalkyl, or —C(H)₂—X—(R³)_{q}—R⁴;
each R³ is independently —S(O₂)—, —CR′₂—, —N(R″)—, —S—, or —O—;
each R⁴ is independently C₁₋₆ fluoroalkyl;
each R′ is independently hydrogen, halo, optionally substituted alkyl, or optionally substituted haloalkyl;
each R″ is independently hydrogen, optionally substituted alkyl, or optionally substituted haloalkyl;
each X is independently —O—, —N(R″)—, or —S—;
each m, p, and q are independently 0, 1, 2, 3, 4, 5, 6, 7, or 8; and
n is an integer between 5 and 30.

39. The compound of claim 82, wherein m is zero.

40. (canceled)

41. The compound of claim 38, wherein m is 5 and —(R¹)ₘ— is —CH₂—CH₂—N(H)—C(O)—O—.

42-52. (canceled)

53. The compound of claim 38, wherein the compound of Formula I is selected from the group consisting of: 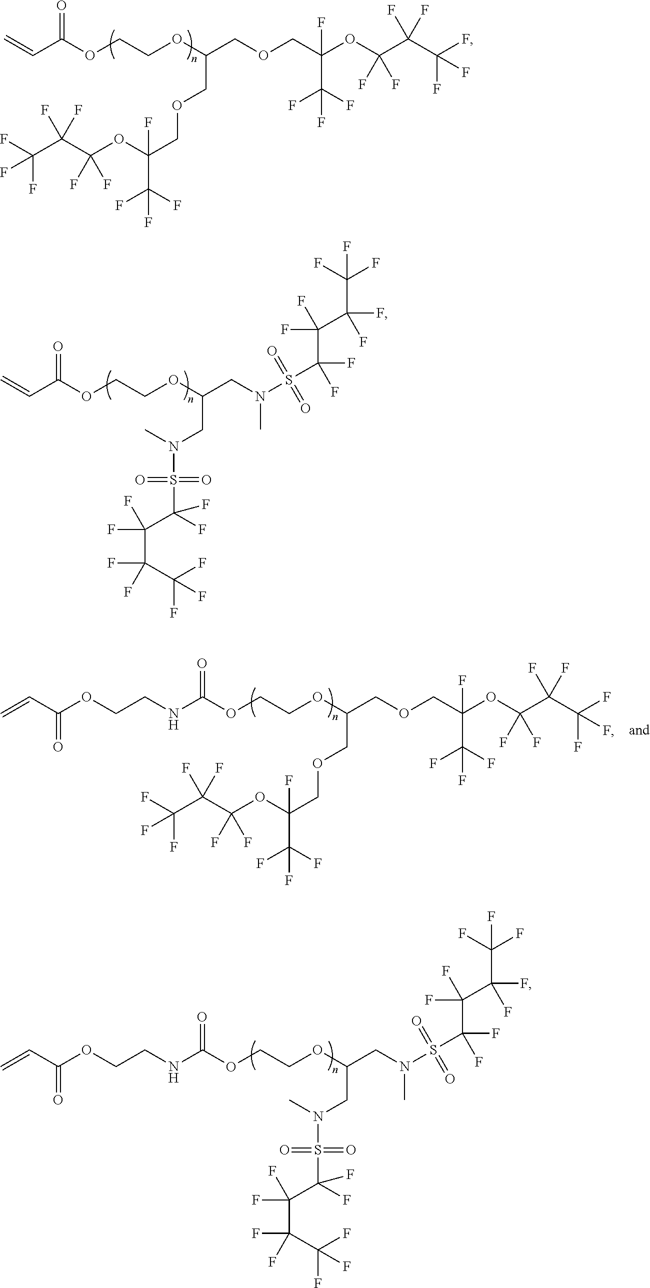
wherein n is as described herein.

54-66. (canceled)

67. The composition of claim 32, wherein some of the multi-acrylate monomers comprise ethylene glycol repeat units.

68-78. (canceled)
